(19) United States
(12) Patent Application Publication
(10) Pub. No.: US 2005/0233435 A1
(43) Pub. Date: Oct. 20, 2005

(54) Plasmodium axenic liver stages as a noninfectious whole organism malaria vaccine

(75) Inventors: Stefan H.I. Kappe, Seattle, WA (US); Victor Nussenzweig, New York, NY (US); Karine Kaiser, New York, NY (US); Nelly Camargo, Seattle, WA (US); Agam P. Singh, New York, NY (US)

Correspondence Address: DARBY & DARBY P.C., P. O. BOX 5257, NEW YORK, NY, 10150-5257, US

(73) Assignee: NYU Medical Center, New York, NY (US)

(21) Appl. No.: 10/936,786

(22) Filed: Sep. 7, 2004

Related U.S. Application Data
(60) Provisional application No. 60/500,793, filed on Sep. 4, 2003.
(60) Provisional application No. 60/540,424, filed on Jan. 29, 2004.

(51) Int. Cl. C12N 1/10; A61K 39/015

(52) U.S. Cl. 435/258.2

(57) ABSTRACT

The present invention relates to the treatment and prevention of malaria infection. In particular, the present invention provides novel noninfectious, whole organism vaccines for malaria, which vaccines comprise a Plasmodium axenic liver stage. The invention also provides methods to treat and prevent malaria by administering such Plasmodium axenic liver stage vaccines, as well as methods to generate Plasmodium axenic liver stages.

### PRIORITY

This application claims priority under 35 U.S.C. § 119(e) to U.S. Provisional Application Ser. No. 60/500,793 filed Sep. 4, 2003 and Ser. No. 60/540,424 filed Jan. 29, 2004 which are incorporated by reference herein in their entirety.

### GOVERNMENT SUPPORT

This work was supported in part by NIH R01 Grant No. AI053709-001 “New Invasion-Related Proteins of Plasmodium Sporozoites”, Dr. Stefan Kappe. Pursuant to the terms of that grant, the federal government may have certain rights to this invention.

### FIELD OF THE INVENTION

The present invention relates to the treatment and prevention of malaria infection. In particular, the present invention provides novel noninfectious, whole organism vaccines for malaria, which vaccines comprise a Plasmodium axenic liver stage substantially free of hepatocyte proteins. The invention also provides methods to treat and prevent malaria by administering such Plasmodium axenic liver stage vaccines, as well as methods to generate Plasmodium axenic liver stages.

### BACKGROUND OF THE INVENTION

Malaria is caused by obligate intracellular protozoan parasites of the genus Plasmodium. Plasmodia have a complex life cycle that alternates between mosquito vector and vertebrate host. Sporozoites are the transmission stage of the Plasmodium parasite that causes malaria. They reside in the salivary glands of mosquitoes. Transmission occurs when infected mosquitoes bloodfeed and inoculate sporozoites. From the skin, sporozoites enter the bloodstream and are quickly transported to the liver where they extravasate and subsequently invade hepatocytes. Within host hepatocytes further development takes place, during which the elongate sporozoites transform into spherical liver stages, also known as exoerythrocytic forms (EEFs).

The liver stages (EEFs) are the obligatory tissue stage intermediary between the parasites' residence in the mosquito vector and the initiation of red blood cell infection by merozoites in the mammalian host (Shortt and Garnham Nature 1948; 161:126). Transformation of sporozoites into liver stages (EEFs) is a radical cellular remodeling process (Meis et al. Nature 1983; 302:424-426). It becomes first discernible by a bulbous expansion of the sporozoite that contains the single nucleus. This bulb progressively enlarges until the banana-shaped sporozoite is completely absorbed. The spherical liver stages (EEFs) then grow, undergo multiple rounds of nuclear division (Meis et al. Cell Tissue Res 1985; 241:353-360) and finally differentiate into thousands of first generation merozoites. Thus, a few days following sporozoite invasion, thousands of merozoites enter the blood and invade red blood cells, where they continue the cycle and produce the typical symptoms of malaria infection.

Malaria is a devastating parasitic disease infecting 300-500 million and killing 2 million people each year, mostly young children in Africa. It is also the number one infectious threat facing the US soldier, and is the leading cause of all casualties during tropical deployments. In addition, malaria poses a great danger to tourists traveling in tropical countries. The Plasmodium parasites causing malaria become increasingly resistant to the small arsenal of available drugs, and there is no malaria vaccine. Thus, the situation is likely to worsen in the years to come.

Malaria vaccines may prevent malaria disease either by generating antibodies that neutralize sporozoite infectivity, or by eliciting T-cells that eliminate the liver stages. Current candidate malaria vaccines contain malaria peptides, recombinant proteins or vectors, or DNA encoding malarial antigens. None have been effective enough to warrant commercial development.

For example, malaria vaccines containing epitopes of the major surface protein of sporozoites (circumsporozoite protein or CSP) are undergoing human trials (Kester et al. J. Infect. Dis. 2001; 183:640-647 and Bojang et al. Lancet. 2001; 358:1927-1934). However, high concentrations of antibodies are required to eliminate all sporozoites, because the parasites remain free in the circulation for only minutes before entering the hepatocytes.

Similarly, an experimental vaccine that consists of gamma irradiated sporozoites that elicits protective immunity in humans and animal models has been described (Hoffman and Doolan. Nature Med. 2000; 6:1218-1219; Nussenzweig and Nussenzweig. Adv Immunol. 1989; 45:283-334; and Hoffman et al. J Infect Dis. 2002; 185:1155-1164). The injected gamma irradiated parasites infect hepatocytes and transform into liver stages, but cannot proliferate (Sigler et al. Am J Trop Med Hyg. 1984; 33:544-547 and Nussler et al. Trop Med Parasitol. 1989; 40:468-469). It is the antigens expressed in the early liver stages that confer the protective immunity observed with this vaccine (Ferreira et al. Science. 1986; 232:881-884). However, the dose of irradiation is very critical for the success of the experimental vaccine. A low dose will not prevent DNA replication, further liver stage development, and onset of malaria disease. A high dose of irradiation will kill the parasites and prevent early development, thereby preventing priming of the protective T-cells. Because of these difficulties, it has never been considered feasible to develop irradiated sporozoites into a vaccine.

Thus, a need exists in the art for a vaccine that can reliably be used to provide protective immunity against malaria infection. In particular, a vaccine that targets the intracellular liver stages of Plasmodium is highly desirable.

It is not known what signals trigger transformation into liver stages nor what factors are essential to support it. Plasmodium liver stages are exceedingly difficult to study as they are rare and preparations are always contaminated with hepatocyte material. Therefore, little is known about their antigenic repertoire with but a few EEF-expressed proteins identified (Hollingdale and Krzych. Chem Immunol 2002; 80:97-124). However, defining antigens expressed in early liver stages is an important goal for pre-erythrocytic vaccine development (Hoffman and Doolan. Nature Med 2000; 6:1218-1219).

Complete development of liver stages of P. berghei, a model Plasmodium species that infects rodents, has been achieved in vitro using the hepatoma cell line HepG2 as a host cell (Hollingdale et al. Am J Trop Med Hyg. 1983; 32:682-684). This in vitro system, together with a line of P. berghei that expresses green fluorescent protein (GFP) (Natarajan et al. Cell Microbiol 2001; 3:371-379), facilitates microscopic monitoring of invasion and subsequent liver stage development.

The present invention relates to the discovery that sporozoites can be cultured so as to transform into liver stages (EEFs) in the absence of host hepatocytes. Thus, the present inventors have discovered a new methodology to obtain Plasmodium liver stages axenically (i.e., without any host hepatocytes). This improvement allows the production of a pure population of Plasmodium liver stages substantially free of contaminating hepatocytes proteins. Furthermore, the present inventors have demonstrated that Plasmodium axenic liver stages elicit a potent protective response against malaria infection. The Plasmodium axenic liver stages are not infective but retain immunogenicity and the ability to elicit protection.

Numerous references, including patents, patent applications, and various publications are cited and discussed in the description of this invention. The citation and/or discussion of such references is provided merely to clarify the description of the present invention and is not an admission that any such reference is “prior art” to the present invention. All references cited and discussed in this specification are incorporated herein by reference in their entirety and to the same extent as if each reference was individually incorporated by reference.

### SUMMARY OF THE INVENTION

The present invention is directed to a method for the generation of a Plasmodium axenic liver stage, which method comprises culturing a Plasmodium sporozoite in the absence of a hepatocyte or a hepatocyte-derived factor, wherein the Plasmodium sporozoite is cultured at a temperature of between about 35° C. and about 39° C. in culture medium comprising between about 5% and about 15% serum. In a preferred embodiment, the cells are cultured for between about 18 and about 24 hours, in a temperature of about 37° C., and the medium comprises about 10% fetal bovine serum.

The invention is also directed to a Plasmodium axenic liver stage produced by the method for the generation of a Plasmodium axenic liver stage. In preferred embodiments, the Plasmodium axenic liver stage is a Plasmodium falciparum axenic liver stage. Plasmodium falciparum is the main cause of human malaria.

The invention is further directed to a method for the prevention or treatment of malaria in a subject in need of such prevention or treatment, which method comprises administering to the subject a vaccine comprising a Plasmodium axenic liver stage. In preferred embodiments of these methods, the Plasmodium axenic liver stage is a Plasmodium falciparum axenic liver stage. In preferred embodiments of these methods, the subject is a human.

The invention is directed to a vaccine for the prevention or treatment of malaria comprising a Plasmodium axenic liver stage and a pharmaceutically acceptable carrier. The vaccines of the invention may further comprise an adjuvant. In preferred embodiments of the vaccines of the invention, the Plasmodium axenic liver stage is a Plasmodium falciparum axenic liver stage.

### DESCRIPTION OF THE DRAWINGS

FIG. 1 depicts the structure of the endogenous CS locus of the Plasmodium berghei NK65 strain that expresses green fluorescent protein during the pre-erythrocytic stages, including the sporozoite and liver stages. “GFP” is the structural gene for the FACS-adapted green fluorescent protein mutant 2, GFPmut2. “5′CS” is the 5′ UTR flanking sequences of the circumsporozoite protein gene. “3′CS” is the 3′ UTR flanking sequences of the circumsporozoite protein gene. “DHFR-TS” is the P. berghei DHFR-TS selection cassette that confers resistance to pyrimethamine. “CSP” is the endogenous sequences of the CS gene that encode the circumsporozoite protein. The shaded boxes indicate sequences derived from the GFPmut2 expression cassette. The hatched boxes indicate sequences of the drug selection cassette including pUC cloning vector-derived sequences. The open boxes indicated endogenous P. berghei genomic sequences. Restriction enzyme sites are indicated. Adapted from Natarajan et al. Cell Microbiol 2001; 3:371-379.

FIG. 2 (A-D) depicts data showing that transformation of Plasmodium sporozoites into exoerythrocytic forms (EEFs) does not require host cells. This figure shows micrographs of GFP fluorescence images from P. berghei transformed EEFs expressing green fluorescent protein. Scale bars are 10 μm. FIG. 2A. Transformed EEFs formed from sporozoites after 24 hours of culture without host cells. FIG. 2B. Transformed EEFs formed from sporozoites after 24 hours of culture with host HepG2 cells. FIG. 2C. Higher magnification of a single transformed EEF formed from a sporozoite after 24 hours of culture without host cells. FIG. 2D. Higher magnification of a single transformed EEF formed from a sporozoite after 24 hours of culture with host HepG2 cells.

FIG. 3 (A and B) depicts data showing that exoerythrocytic forms (EEFs) formed by culture of Plasmodium sporozoites without host cells express key EEF-specific antigens. FIG. 3A. Immunoblot analysis of HSP70 expression. Sporozoites (Spz) express HSP70 at barely detectable levels. EEFs (EEF), derived from host-cell-free culture of sporozoites for 24 hours, show a dramatic increase in HSP70 expression. The sizes of molecular weight standards, in kilodaltons (kDa), are indicated. FIG. 3B. Reverse transcriptase (RT)-PCR with gene-specific oligonucleotide primers amplified specific fragments of 120 base pairs (bp). EEFs, derived from host-cell-free culture of sporozoites for 24 hours, express transcripts encoding the merozoite surface protein-1 (MSP-1, left panel) and the hepatic and erythrocytic stage protein 17 (HEP17, right panel). “+RT” are experimental reactions performed with reverse transcription. “−RT” are control reactions without prior reverse transcription. The size of molecular weight standards, in base pairs (bp), is indicated.

FIG. 4 (A and B) depicts data showing that the inner membrane complex (IMC) is lost during host cell-free development of transformed exoerythrocytic forms (EEFs). EEFs were stained with antibodies against myosin A tail domain interacting protein (MTIP), an IMC marker. FIG. 4A. An EEF generated by 24 hours of culture without host cells. FIG. 4B. An EEF generated by 24 hours of culture with HepG2 host cells.

FIG. 5 depicts data showing ribosomal RNA (rRNA) type switch in EEFs generated by 24 hour host cell-free culture. RT-PCR with type-specific primers was used to detect expression of A-type rRNA (left panel) or S-type rRNA (right panel) in cultures grown at 37° C. (37° C.) or at 22° C. (22° C.). “−RT” is a negative control reaction without prior reverse transcription for 37° C. A-type and 22° C. S-type.

FIG. 6 depicts data showing that vaccination with Plasmodium axenic liver stages confers protective immunity against malaria infection. Groups of mice were immunized three times with the indicated dose (X-axis) of live Plasmodium yoelii axenic liver stages. Note that the “Injected doses” are given as total parasites/transformed parasites, where “total parasites” indicates the number of sporozoites inoculated into the host-cell free transformation culture and “transformed parasites” figure indicates the dose of live Plasmodium yoelii axenic liver stages obtained following host-cell free transformation culture. “1 M” is one million (1,000,000). Seven days after the last immunization, the mice were challenged by intravenous injection of 10,000 infectious Plasmodium yoelii sporozoites. Forty-eight hours later, the livers of challenged mice were collected, and the liver-stage burden determined. Relative inhibition (in percentage, Y-axis) of liver-stage burden for immunized mice was calculated relative to the liver-stage burden of naïve mice (Control). Bars represent an average of relative inhibition values calculated from the indicated number of immunized mice in each dose group (sample size, n). Error bars represent the standard deviation for each dose group.

### DETAILED DESCRIPTION

Malaria parasite species that infect mammals, including humans, must first take up residence in hepatic host cells as liver stages (exoerythrocytic forms, EEF) before initiating the infection of red blood cells that leads to malaria disease. Despite the importance of liver stages for immunity against malaria, little is known about their biology and antigenic composition.

The present invention is based upon the unexpected discovery that sporozoites, the transmission stage of the malarial parasite that resides in the mosquito vector salivary glands, can transform into liver stages (EEFs) without intracellular residence in host hepatocytes. The morphological sequence of transformation and the expression of proteins in these Plasmodium axenic liver stages appear indistinguishable from liver stages that develop within host hepatocytes. Axenic transformation depends on temperature elevation to about 37° C. and serum. These novel findings demonstrate that residence in a host hepatocyte or specific host cell-derived factors are not necessary to bring about the profound morphological and biochemical changes of the parasite that occur after its transmission from vector to mammalian host. Thus, the present inventors have discovered a new methodology to obtain Plasmodium liver stages axenically (i.e., without any host hepatocytes) with the result the Plasmodium liver stages can be reliably cultured substantially free of hepatocyte proteins.

The present inventors have further demonstrated that immunization with such Plasmodium axenic liver stages provides protective immunity against malarial infection. The Plasmodium axenic liver stages are not infective but retain immunogenicity and the ability to elicit protection. Thus, the present invention provides an effective attenuated, non-infectious malaria vaccine comprising Plasmodium axenic liver stages substantially free of hepatocyte proteins, as well as methods of preventing and treating malaria comprising administering such vaccines.

### Definitions

As used herein, the term “Plasmodium liver stage” or “liver stage” refers to the spherical exoerythrocytic form (EEF) of the malaria parasite, which develops in vivo by transformation of sporozoites found intracellularly within host hepatocytes. These liver stages (or EEFs) are the obligatory tissue stage intermediary between the parasites' residence in the mosquito vector and the initiation of red blood cell infection by merozoites in the mammalian host. Transformation of sporozoites into liver stages (EEFs) is first discernible by a bulbous expansion of the sporozoite that contains the single nucleus. This bulb progressively enlarges until the banana-shaped sporozoite is completely absorbed. The spherical liver stages (EEFs) then grow, undergo multiple rounds of nuclear division, and eventually differentiate into thousands of first generation merozoites.

As used herein, the term “Plasmodium axenic liver stage” refers to a liver stage (or EEF) of the malaria parasite that has transformed without being exposed to host hepatocytes, (e.g., either by invading hepatocytes, or by exposure to hepatocytes or hepatocyte-derived factors). Plasmodium axenic liver stages are generated by culturing sporozoites, in the absence of hepatocytes or hepatocyte-derived factors, at between about 35° C. and about 39° C., preferably at about 37° C., in medium containing serum. This stage can be identified by assay for stage-specific markers such as HSP70, CSP, TRAP, MTIP, and HEP17. “Substantially free” preparations are defined herein as without detectable hepatocytes or hepatocyte surface markers. Plasmodium axenic liver stage, therefore, is substantially free from detectable hepatocyte proteins or hepatocyte surface markers.

As used herein, the term “hepatocyte-derived factor” refers to a cellular product produced by a hepatocyte, which cellular product is present in cell-free culture medium isolated from an in vitro culture of hepatocytes. A Plasmodium sporozoite is cultured “in the absence of a hepatocyte or a hepatocyte-derived factor” where the sporozoite culture does not comprise a hepatocyte, or a hepatocyte derived factor.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

As used herein, the term “vaccine” refers to a composition comprising an antigen, and optionally other pharmaceutically acceptable carriers, administered to stimulate an immune response specifically against the antigen and preferably to engender immunological memory that leads to mounting of a protective immune response should the subject encounter that antigen at some future time. In the vaccines of the present invention, the antigen comprises a Plasmodium axenic liver stage. Vaccines often comprise an adjuvant.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition in the host, symptoms of malaria.

As used herein, the term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, BCG (bacille Calmette-Guerin) and Corynebacterium parvum. Preferably, the adjuvant is pharmaceutically acceptable.

The following abbreviations are used herein: EEF, exoerythrocytic form; TRAP, thrombospondin-related anonymous protein; CSP, circumsporozoite protein; IMC, inner membrane complex; MTIP, MyoA tail domain interacting protein; GFP, green fluorescent protein; MSP-1, merozoite surface protein-1; and HEP17, hepatic and erythrocytic stage protein 17.

### Method to Obtain Plasmodium Axenic Liver Stages

The present invention provides methods to obtain Plasmodium axenic liver stages by culturing Plasmodium sporozoites in vitro in the absence of hepatocyte host cells. The methods to obtain Plasmodium axenic liver stages comprise culturing a Plasmodium sporozoite in the absence of hepatocytes or hepatocyte-derived factors, wherein the Plasmodium sporozoite is cultured at a temperature of about 37° C. in culture medium comprising serum. Experiments have shown that elevated temperature (about 37° C.) and the presence of serum in the culture medium are necessary to obtain complete transformation of a sporozoite to a mature Plasmodium axenic liver stage. Temperatures ranging between about 35° C. and about 39° C. are preferred in this invention. Serum concentrations ranging between about 5% and about 15% are also preferred in this invention. Culture for between about 18 and about 30 hours are preferred.

Experiments have further shown that culture for up to 24 hours at reduced temperature (about 22° C.) prior to culture at elevated temperature (about 37° C.) also results in the transformation of sporozoites to Plasmodium axenic liver stage. Thus, the present invention also encompasses variations of the method wherein the Plasmodium sporozoites are cultured at reduced temperature (about 22° C.) prior to the culture at elevated temperature (about 37° C.). In fact, such a culture scheme may result in a greater overall transformation yield (expressed as % of cultured sporozoites that transform into Plasmodium axenic liver stages). Reduced temperatures between about 18° C. and about 24° C., and elevated temperatures between about 35° C. and about 39° C. are provided in this invention. Serum concentrations between about 5% and about 15% are also preferred in this invention. Culture at a reduced temperature for up to about 24 hours and culture at an elevated temperature for between 18 and about 30 hours are preferred.

In a preferred embodiment, the medium comprises between about 5% and about 15%, most preferably about 10%, by volume of fetal bovine serum. The invention also contemplates the use of other sources of serum, including fetal horse serum, fetal sheep serum, fetal pig serum, and guinea pig serum. Serums derived from various sources are commercially available (for example from HyClone or Gibco).

In a preferred embodiment, DMEM is the culture media. However, other non-limiting examples of commercially available culture media which can also be suitable for use in the present invention are adherent cell medias such as MEM or M-199, suspension cell media such as RPMI-1640 and derivatives thereof. One of ordinary skill in the art will be able to ascertain substitute media for the growth of Plasmodium axenic liver stages. Such media is available from numerous sources such as Gibco, Invitrogen, Hyclone, and SigmaAldrich.

In experiments, it was noted that complete transformation of a cultured sporozoite into a Plasmodium axenic liver stage could be observed after about 18 hours of culture at a temperature of about 37° C. Furthermore, after about 24 hours of culture at a temperature of about 37° C., no un-transformed sporozoites remained in the culture. Thus, the remaining viable parasites were all Plasmodium axenic liver stages. Therefore, by culturing Plasmodium sporozoites in vitro in the absence of hepatocyte host cells for about 24 hours, a pure population of Plasmodium axenic liver stages may be obtained. Such pure populations are particularly advantageous for use in formulating the vaccines of the invention, as they no longer contain infective sporozoites.

The absence of infective sporozoites in a population of Plasmodium axenic liver stages may be confirmed by various techniques, including evaluation of parasite morphology, for example by microscopy, and evaluation of stage-specific gene expression via immunoassay, Northern blot, in situ hybridization, RT-PCR, or other techniques well known in the art.

For example, the morphology of a malarial parasite may be determined by standard phase contrast microscopy, wherein a sporozoite will show an elongated banana-shaped morphology while a liver-stage will show the characteristic spherical morphology.

A variety of stage-specific markers are known and may be used to confirm parasite stage. For example, heat shock protein 70 (HSP70) of Plasmodium is highly expressed in liver stages and erythrocytic stages of the parasite, but is barely detectable in sporozoites (Bianco et al. Proc Natl Acad Sci USA 1986; 83:8713-8717; Tsuji et al. Parasitol Res 1994; 80:16-21; and Kumar et al. Parasitol Res 1993; 79:109-113). HSP70 expression can therefore be used to follow the transformation of sporozoites into liver stages. In contrast, TRAP, a sporozoite-specific micronemal protein involved in host cell invasion (Sultan et al. Cell 1997; 90:511-522), is highly expressed in sporozoites, but not detectable in liver stages. The loss of TRAP expression coincides with the disappearance of micronemes after sporozoite transformation in vivo (Meis et al. Cell Tissue Res 1985; 241:353-360). The merozoite surface protein-1 (MSP-1) (Suhrbier et al. Am J Trop Med Hyg 1989; 40:351-355) and the hepatic and erythrocytic stage protein 17 (HEP17) (Charoenvit et al. Exp Parasitol 1995; 80:419-429) are also both found in liver stages, but not in sporozoites. For rodent model parasites, HEP17 is an additional stage-specific marker.

Apicomplexan zoites, including the Plasmodium sporozoite are delimited by a tri-laminar pellicle consisting of a plasma membrane and two closely aligned inner membranes that form the inner membrane complex (IMC). After hepatocyte invasion the sporozoite plasma membrane becomes the EEF plasma membrane, however, the IMC is disassembled and is not detectable at approximately 30 hours post invasion (Meis et al. Cell Tissue Res 1985; 241:353-360). MTIP localizes to the inner membrane complex of sporozoites (Bergman et al. J Cell Sci. 2003; 116:39-49) and can be used as a marker to observe the loss of the IMC during liver stage development.

Switching occurs between the expression of different types of ribosomal RNA (rRNA) at transition points in the Plasmodium life cycle (Gunderson et al. Science 1987; 238:933-937). One such transition is the transformation of sporozoites into liver stages when rRNA expression switches from S-type in sporozoites (S for sporozoite) to A-type in liver stages and succeeding blood stages (A for Asexual) (Zhu et al. J Biol. Chem. 1990; 265:12740-12744). Thus, A-type rRNAs are expressed in liver stages, but not in sporozoites.

The present or absence of these markers in a malaria parasite may be confirmed by a variety of techniques well established in the art, including immunoassays using antibodies specific for the particular marker. Suitable immunoassays include immunoprecipitation, immunoblot, whole mount immunoassay, and enzyme linked immunosorbent assay (ELISA) (see, for example, Ausubel et al., eds. Current Protocols in Molecular Biology, (John Wiley & Sons, Inc.: 1994) and Harlow and Lane. Using Antibodies: A Laboratory Manual (Cold Spring Harbor Laboratory Press: 1999). The expression of these markers in a malaria parasite may also be detected by hybridization-based methods using a nucleotide sequence specific to the marker of interest, including southern blot, dot blot, and in situ hybridization. The expression of these markers may also be determined by RT-PCR conducted using oligonucleotide primers specific to the marker of interest. Such techniques are well described in the art (see, for example, Sambrook, Fritsch and Maniatis, Molecular Cloning: A Laboratory Manual, 3^{rd} Edition (Cold Spring Harbor Laboratory Press, New York:2001); and Ausubel et al., eds. Current Protocols in Molecular Biology, (John Wiley & Sons, Inc.: 1994).

More than 50 years after their discovery (Shortt and Garnham. Nature 1948; 161:126), the biology and antigenic repertoire of liver stages (EEFs) remain largely unstudied, mainly due to the small number if liver stages that can be obtained in vivo and in vitro and the technical challenge of separating the intracellular EEF from uninfected hepatocytes and surrounding host hepatocytes. Conversely, the importance of liver stage antigens for the development for vaccines to confer sterile, protective immunity to malaria has been established. The identification of liver stage antigens is therefore regarded as an important goal in malaria research. Such identification of liver stage antigens will be greatly facilitated by the present invention, in that it makes available for the first time large quantities of isolated Plasmodium liver stages.

This novel method to generate Plasmodium axenic liver stages will, therefore, be extremely useful for many aspects of research into the basic biology of the malaria parasite.

### Vaccines of the Present Invention, and Their Use in Methods to Treat or Prevent Malaria, or to Enhance an Immune Response to Malaria

The present invention provides methods for prevention and treatment of malaria in a subject, which methods comprise administering to the subject an amount effective to treat or prevent malaria of a vaccine comprising a Plasmodium axenic liver stage. The subject to which the vaccine is administered in accordance with these methods may be any human or non-human animal susceptible to infection with the malaria parasite. For such methods, administration can be oral, parenteral, intranasal, intramuscular, intravascular, or any one or more of a variety of well-known administration routes. Moreover, the administration may be by continuous infusion or by single or multiple boluses.

The prevention and/or treatment of malaria may be readily ascertained by the skilled practitioner by evaluation of clinical symptoms associated with malarial infection, for example percent parasitism of red blood cells, fatigue, and comatose state. Thus, according to the methods of the present invention, the subject shows improved or absent clinical symptoms of malaria infection following administration of a vaccine comprising a Plasmodium axenic liver stage.

Generating an immune response in a subject can be measured by standard tests including, but not limited to, the following: direct measurement of peripheral blood lymphocytes by means known to the art; natural killer cell cytotoxicity assays (Provinciali et al (1992) J. Immunol. Meth. 155: 19-24), cell proliferation assays (Vollenweider et al. (1992) J. Immunol. Meth. 149: 133-135), immunoassays of immune cells and subsets (Loeffler et al. (1992) Cytom. 13: 169-174; Rivoltini et al. (1992) Can. Immunol. Immunother. 34: 241-251); and skin tests for cell mediated immunity (Chang et al. (1993) Cancer Res. 53: 1043-1050). For an excellent text on methods and analyses for measuring the strength of the immune system, see, for example, Coligan et al. (Ed.) (2000) Current Protocols in Immunology, Vol. 1, Wiley & Sons.

The invention also encompasses therapeutically effective amounts of vaccines as defined herein. The vaccines of the invention comprise therapeutically effective amounts of a Plasmodium axenic liver stage substantially free of hepatocytes proteins and a pharmaceutically acceptable carrier. Methods of formulating pharmaceutical compositions and vaccines are well-known to those of ordinary skill in the art (see, e.g., Remington's Pharmaceutical Sciences, 18^{th} Edition, Gennaro, ed. (Mack Publishing Company: 1990)).

Such vaccines may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in dosage forms appropriate for each route of administration. In general, comprehended by the invention are vaccines comprising a Plasmodium axenic liver stage, together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. See, e.g., Remington's Pharmaceutical Sciences, 18th Ed. (1990, Mack Publishing Co., Easton, Pa. 18042) pages 1435-1712 which are herein incorporated by reference.

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets, pellets, powders, or granules. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include the therapeutic agent and inert ingredients which allow for protection against the stomach environment, and release of the biologically active material in the intestine.

Also contemplated for use herein are liquid dosage forms for oral administration, including pharmaceutically acceptable emulsions, solutions, suspensions, and syrups, which may contain other components including inert diluents; adjuvants, wetting agents, emulsifying and suspending agents; and sweetening, flavoring, coloring, and perfuming agents.

For oral formulations, the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the therapeutic agent beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used. The formulation of the material for capsule administration could also be as a powder, lightly compressed plugs, or even as tablets. These therapeutics could be prepared by compression.

One may dilute or increase the volume of the therapeutic agent with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic agent into a solid dosage form. Materials used as disintegrates include but are not limited to starch, including the commercial disintegrant based on starch, Explotab, Sodium starch glycolate, Amberlite, sodium carboxymethylcellulose, ultramylopectin, sodium alginate, gelatin, orange peel, acid carboxymethyl cellulose, natural sponge and bentonite may all be used. The disintegrants may also be insoluble cationic exchange resins. Powdered gums may be used as disintegrants and as binders and can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the peptide (or derivative).

An antifrictional agent may be included in the formulation to prevent sticking during the formulation process. Lubricants may be used as a layer between the therapeutic agent and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the therapeutic agent during formulation and to aid rearrangement during compression might be added. The glidants may include starch, talc, pyrogenic silica and hydrated silicoaluminate.

To aid dissolution of the therapeutic agent into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Controlled release oral formulations may be desirable. The therapeutic agent could be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release is by a method based on the Oros therapeutic system (Alza Corp.), i.e. the therapeutic agent is enclosed in a semipermeable membrane which allows water to enter and push agent out through a single small opening due to osmotic effects.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid. A mix of materials might be used to provide the optimum film coating. Film coating may be carried out in a pan coater or in a fluidized bed or by compression coating.

In the case of vaccines, it is often observed that a primary challenge with an antigen alone, in the absence of an adjuvant, will fail to elicit a humoral or cellular immune response. Therefore the vaccines of the invention may contain adjuvants including, but not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, BCG (bacille Calmette-Guerin) and Corynebacterium parvum. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Where the vaccine is intended for use in human subjects, the adjuvant should be pharmaceutically acceptable.

Vaccines according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants, preserving, wetting, emulsifying, and dispersing agents. They can also be manufactured using sterile water, or some other sterile injectable medium, immediately before use.

In order to determine the effective amount of the vaccines of the present invention, the ordinary skilled practitioner, considering the therapeutic context, age, and general health of the recipient, will be able to ascertain proper dosing. The selected dosage depends upon the desired therapeutic effect, on the route of administration, and on the duration of the treatment desired. The dosing schedule may vary, depending on the circulation half-life and the formulation used. Experiments to determine levels for dosages can be ascertained by one of ordinary skill in the art by the use of animal models of malaria well known to those of ordinary skill in the art which are predictive of such as described in Example 2 or a model of human malaria wherein Aotus monkeys are infected by Plasmodium Falciparum (Jones et al. (2000) Am. J. Trop. Med. Hyg., 62: 675-680) which are predictive of the human immune response.

The vaccines of the present invention may be administered in conjunction with one or more additional active ingredients, pharmaceutical compositions, or vaccines.

### EXAMPLES

The present invention is next described by means of the following examples. However, the use of these and other examples anywhere in the specification is illustrative only, and in no way limits the scope and meaning of the invention or of any exemplified form. Likewise, the invention is not limited to any particular preferred embodiments described herein. Indeed, many modifications and variations of the invention may be apparent to those skilled in the art upon reading this specification, and can be made without departing from its spirit and scope. The invention is therefore to be limited only by the terms of the appended claims, along with the full scope of equivalents to which the claims are entitled.

In accordance with the present invention there may be employed conventional molecular biology, microbiology, protein expression and purification, antibody, and recombinant DNA techniques within the skill of the art. Such techniques are explained fully in the literature. See, e.g., DNA Cloning: A Practical Approach, Volumes I and II (Glover ed.: 1985); Oligonucleotide Synthesis (Gait ed.: 1984); Nucleic Acid Hybridization (Hames & Higgins eds.: 1985); Transcription And Translation (Hames & Higgins, eds.: 1984); Animal Cell Culture (Freshney, ed.: 1986); Immobilized Cells And Enzymes (IRL Press: 1986); Perbal, A Practical Guide To Molecular Cloning (1984); Ausubel et al., eds. Current Protocols in Molecular Biology, (John Wiley & Sons, Inc.: 1994); Sambrook et al. Molecular Cloning: A Laboratory Manual, Third Edition (Cold Spring Harbor Laboratory Press: 2001); Harlow and Lane. Using Antibodies: A Laboratory Manual (Cold Spring Harbor Laboratory Press: 1999); PCR Primer: A Laboratory Manual, Second Edition. Dieffenbach and Dveksler, eds. (Cold Spring Harbor Laboratory Press: 2003); and Hockfield et al. Selected Methods for Antibody and Nucleic Acid Probes (Cold Spring Harbor Laboratory Press: 1993).

### Example 1

### Generation of Plasmodium Axenic Liver Stages in Host Cell-Free Culture

### Materials and Methods

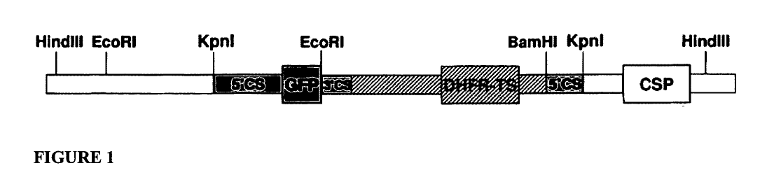

GFP-expressing Plasmodium berghei NK65: The construction of a Plasmodium berghei line that expresses green fluorescent protein during the pre-erythrocytic stages, including the sporozoite and liver stages, has been described (Natarajan et al. Cell Microbiol 2001; 3:371-379). In this strain, a cassette containing the structural gene for the FACS-adapted green fluorescent protein mutant 2 (GFPmut2, first described in Cormack et al. Gene 1996; 173:33-38), expressed from the 5′ and 3′ flanking sequences of the circumsporozoite (CS) protein gene, is integrated into the endogenous CS locus of P. berghei strain NK65 (see FIG. 1).

Malaria parasites: P. berghei belongs to a group of four Plasmodium species that infect murine rodents. These species are P. vinckei, P. chabaudi, P. yoelii and P. berghei. These parasites have proved to be analogous to the malarias of man and other primates in most essential aspects of structure, physiology and life cycle and are useful in rodent models of human malaria (Carter and Diggs 1977. In: ‘Parasitic Protozoa’, vol. 3 pp. 359-465. Academic Press, New York).

4-5 day old female Anopheles stephensi mosquitoes were blood-fed on anaesthetized Swiss CD-1/ICR mice that had been infected with wildtype P. berghei strain NK65 or a GFP-expressing P. berghei NK65 line. The mice had been assayed for high levels of parasitemia and the abundance of gametocyte-stage parasites capable of exflagellation. Determination of parasitemia, visualization of malaria parasites in infected blood, and visualization of sporozoites in host mosquitoes was performed as previously described (see, e.g., Sinden et al. Methods Mol Med. 2002; 72: 25-40).

After the infective bloodmeal, mosquitoes were maintained at 21° C. and 80% humidity. On day 10 post-feeding, mosquitoes were dissected in RPMI 1640 medium (Gibco) and isolated midguts were examined for the infection rate. Only mosquito cages having at least 70% of mosquitoes infected were kept for further analysis. Mosquitoes were rinsed in 70% ethanol 5 minutes and washed twice in sterile medium to reduce contamination. Salivary glands were dissected in sterile DMEM medium (BioWhittaker) containing 500 U/ml penicillin/streptomycin and 1.25 μl/ml fungizon (Sigma). The glands were disrupted, sporozoites isolated, and counted in a hemocytometer.

Transformation medium and quantification of transformation: Sporozoites were suspended in DMEM medium (BioWhittaker) containing 2 mM of L-glutamine, 4.5 g/l glucose and supplemented with 10% Fetal Bovine Serum (FBS; HyClone) and 500 U/ml penicillin/streptomycin and 1.25 μl/ml fungizon (Sigma). For the generation of Plasmodium axenic liver stages, 5×10⁴ sporozoites were inoculated per well of a 8 well chamber slide (Labtek) and maintained at 37° C. in 5% CO₂. Initially, slides were coated with Matrigel (Becton Dickinson) at density of 100 μg/cm² to improve the development. Matrigel improved attachment of the parasites to the slide, but was not essential for transformation.

To determine transformation rates for various conditions and slides, the host cell-free cultures of GFP-expressing P. berghei sporozoites were examined by fluorescence microscopy at 400× magnification using an Eclipse TE 300 fluorescence microscope (Nikon, Inc.) using a green filter (excitation wavelength 495 nm/emission wavelength 519 nm). EEFs were scored as completely transformed only when they appeared completely spherical with no sporozoite remnants visible. Each count was performed in triplicate on three independent experiments done with sporozoites isolated from different batches of infected mosquitoes.

As a control to generate EEFs within host hepatocytes, P. berghei sporozoites were cultivated in HepG2-A16 cells (American Type Culture Collection HB 8065). In vitro cultured HepG2 cells were suspended to a cell density of 5×10⁵ cells/mL in DMEM medium (BioWhittaker) containing 2 mM of L-glutamine, 4.5 g/l glucose and supplemented with 10% Fetal Bovine Serum (FBS; HyClone) and 500 U/ml penicillin/streptomycin and 1.25 μl/ml fungizon (Sigma). Then 400 μL of the cell suspension (2×10⁵ HepG2 cells) were added to one well of an 8 well chamber slide, and the cells incubated for 48 hours at 37° C. with 5% CO₂. Next, the culture medium was replaced with fresh medium, and each well was inoculated with 5×10⁴ sporozoites. The sporozoite-inoculated cells were further cultured for 24 hours at 37° C. with 5% CO₂. Intercellular transformation of the sporozoites into EEFs (liver stages) was confirmed by fluorescence microscopy (where GFP-expressing P. berghei strain NK65 was used), or by immunofluorescence assay (performed as described for Plasmodium axenic liver stages below).

Immunofluorescence assays: Transformation of sporozoites into EEFs was confirmed by immunofluorescence assay using monoclonal antibodies against HSP70 (heat shock protein 70) (generation of the antibody as described in Tsuji at al. Parasitol. Res. 1994; 80:16-21); CSP (Circumsporozoite Protein) (generation of the antibody as described in Yoshida et al. Science 1980; 207:71-73); TRAP (Thrombospondin-related anonymous protein) (generation of the antibody as described in Grant et al. Infect Immun. 2000; 68:3667-73) and a rabbit polyclonal antibody against MTIP (Myosin A tail domain interacting protein) an inner membrane complex-associated protein (generation of the antibody as described in Bergman et al. J. Cell. Sci. 2003; 116:39-49).

Parasites were removed from culture chambers, fixed in 2% paraformaldehyde, washed in phosphate buffered saline (PBS), concentrated by centrifugation and suspended in 1% FBS/PBS diluted primary antibody and incubated at 37° C. for 1 hour. After 3 washes, samples were incubated in 6 diamidino-2-phenyllindole (DAPI) at 40 μg/ml for 30 minutes at room temperature. After three washes in 1% FBS/PBS, parasites were transferred onto 12 well slides and dried at room temperature. Slides were incubated with secondary antibodies conjugated with Alexa fluor 488 or Alexa fluor 594 (Molecular Probes) diluted 1:250 in 1% FBS/PBS for 1 hour at 37° C.

Parasites were then visualized using an Eclipse TE 300 fluorescence microscope (Nikon, Inc.) using a green filter (excitation wavelength 495 nm/emission wavelength 519 nm) with a 100× oil APO 1.4 objective.

Immunoblot: 8×10⁵ salivary gland sporozoites were inoculated onto an 8 well chamber slide maintained for 24 hours at 37° C. (as described above). These cultures parasites were then collected. The Transformation rate for this batch was 13%. 8×10⁵ salivary gland sporozoites served as a negative control.

Samples were suspended in 10 μl of sodium-dodecyl-sulfate (SDS) loading buffer and incubated for 5 min at 70° C. Antigen extracts were subjected to 7% SDS-polyacrylamide gel electrophoresis (SDS-PAGE) and transferred onto a PVDF membrane. The membrane was blocked overnight at 4° C. in PBS containing 5% dry milk, and incubated for 1 hour with the anti-HSP70 primary antibody. The membrane was washed in PBS/0.1% Tween 20 and incubated for 1 hour with 1:5000 diluted horseradish peroxidase labeled anti-mouse IgG (Amersham Biosciences) and developed with enhanced chemiluminescence (ECL, Amersham Biosciences), performed according to the manufacturer's instructions.

Reverse transcriptase (RT)-PCR: 5×10⁵ P. berghei sporozoites were inoculated on 12 well-plates and placed at 37° C. or 22° C. 24 hours after inoculation, the contents of each well was collected and total RNA extraction was performed using the RNeasy mini kit (Qiagen), as per manufacturer's instructions. RNA was treated with DNAse I (Invitrogen) to remove contaminating genomic DNA. First strand cDNA synthesis was performed according to manufacturer's instructions using a RT-PCR kit (PE applied Biosciences) with random hexamers. Control reactions were performed in the absence of reverse transcriptase.

PCR amplification was then performed using primers specific for the P. berghei A type rRNA: primers ATYPEsense (5′-CGA GAA TCT TGG CTC CGC CTC G-3′; SEQ ID NO: 1) and ATYPEantisense (5′-CTA AGA AAT CCC CGA AGG GAA ATC-3′: SEQ ID NO: 2), which amplify a specific 460 base pair (bp) fragment. P. berghei S type rRNA was amplified using primers STYPEsense (5′-CAT GAC TTC TGT CAC TGC TTT TAT C-3′; SEQ ID NO: 3) and STYPEantisense (5′-CTA CTC CTT TAA AGA AGA TAG TT-3′; SEQ ID NO: 4), which amplify a specific 457 bp fragment. PCR was performed for 30 cycles of: 95° C. for 15 s, 58° C. for 30 s and 72° C. for 3 min.

PCR amplification for MSP-1 was performed using primers MSPsense (5′-GGA GAA AAT GCA GTG GTA AG-3′; SEQ ID NO: 5) and MSPantisense (5′-TTT TAA ATG CCT CAA GAA TAT TTT TTT CT-3′; SEQ ID NO: 6), which amplify a specific 120 bp fragment. PCR amplification for HEP17 was performed using primers HEPsense (5′-AAA AGG GAA GAC ATC CAT TC-3′; SEQ ID NO: 7) and HEPantisense (5′-TGT ATT CCT TCG GAT GAA AAA-3′; SEQ ID NO: 8), which amplify a specific 120 bp fragment. PCR was performed for 30 cycles of: 95° C. for 15 s, 55° C. for 30 s and 72° C. for 3 min.

### Results and Discussion

Sporozoites were cultured without HepG2 cells in micro-chamber slides coated with basement membrane extract (Matrigel) and DMEM medium containing 10% fetal bovine serum (FBS) for 4, 10 and 24 hours at 37° C. The P. berghei strain used expresses green fluorescent protein in sporozoites and in the liver stages, enabling the visualization of live parasites by fluorescence microscopy.

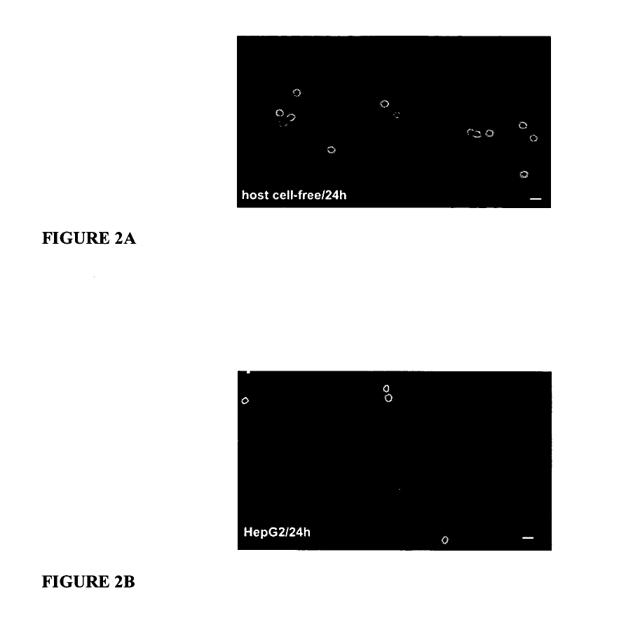

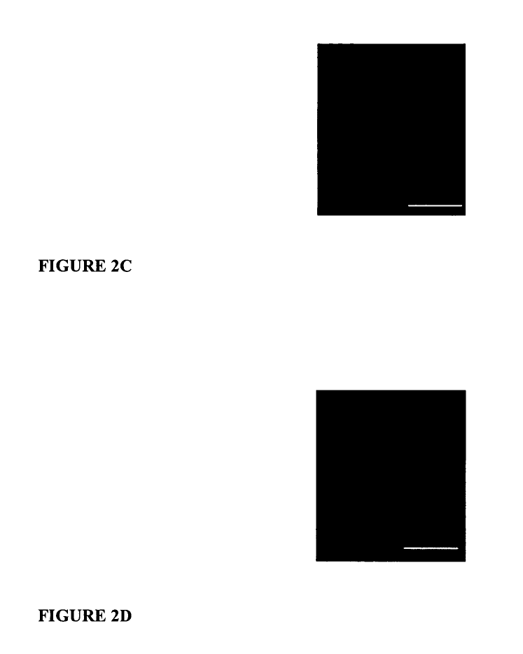

Microscopic examination showed that after 4 hours of culture at 37° C. in 10% FBS sporozoites frequently developed the transformation bulb that was also typically observed during early intracellular transformation (Meis et al. Nature 1983; 302:424-426). At 10 hours the extracellular transformation had proceeded further to intermediate EEF-like forms displaying a more extended bulb and further retraction of the arm-like remnants of the sporozoite cell body, closely resembling the progression of transformation observed during intracellular transformation (Meis et al. Cell Tissue Res 1985; 241:353-360 and Hollingdale Am J Trop Med Hyg. 1983; 32:685-690. At 24 hours ˜13% of sporozoites had transformed into completely spherical EEF-like forms (FIG. 2A, FIG. 2C and Table 1) that are indistinguishable from EEFs that developed within host cells (FIG. 2B and FIG. 2D).

This rate of transformation exceeds the published rates of transformation achieved in HepG2 cells which range from 3-8% (Hollingdale et al. Am J Trop Med Hyg 1983; 32:682-684 and Calvo-Calle et al. Exp Parasitol 1994; 79:362-373) and the rates of transformation in HepG2 cells observed in our control experiments (˜5%).

EEF-like forms that were cultured without HepG2 cells were similar in size (6-10 μm) and morphology when compared to EEFs that grew within HepG2 cells (FIG. 2C and FIG. 2D). No sporozoites were detectable in 24 hour, host cell-free cultures (see FIG. 2A), indicating that untransformed sporozoites did not survive.

These results show that transformation of Plasmodium sporozoites into exoerythrocytic forms (EEFs) does not require host cells. Sporozoites cultured without HepG2 cells at 37° C. in 10% FBS transform through the classic sequence of morphological changes into liver stages (EEFs).

TABLE 1
Host cell-free transformation of culture sporozoites into liver stages

| culture conditions^{§} | EEFs* | transformation^{†} |
|---|---|---|
| 37° C. | 6725.34 | 13.43% |
| 22° C. | 34.67 | 0.07% |
| 22° C. → 37° C. | 9656 | 19.32% |
| 37° C. w/o 10% FBS | 346.67 | 0.69% |

^{§}24 hours cultures except 22° C. → 37° C. shift (48 hours)
*Average number from 3 independent experiments.
^{†}Percentage of transformation calculated based on the 5 × 10⁴ sporozoites used for culture

Expression of EEF proteins was comparable between EEFs that undergo intracellular development and EEFs that develop without host cells, using antibodies against heat shock protein 70 (HSP70), circumsporozoite protein (CSP), thrombospondin-related anonymous protein (TRAP) and myosin A tail domain interacting protein (MTIP).

HSP70 of Plasmodium is highly expressed in EEF and erythrocytic stages of the parasite, it is however barely detectable in sporozoites (Bianco et al. Proc Natl Acad Sci USA 1986; 83:8713-8717; Tsuji et al. Parasitol Res 1994; 80:16-21; and Kumar et al. Parasitol Res 1993; 79:109-113). HSP70 expression can therefore be used to follow the transformation of sporozoites into liver stages.

After 6 hours of host cell-free culture, early extracellular EEF-like forms identified by the transformation bulb showed increasing HSP70 expression. At this stage, the HSP70 expression is mostly localized to the bulb. After 18-24 hours of culture, completely spherical EEF-like forms had developed that showed intense HSP70 staining. When cultures were incubated for 48 hours most of these early EEF-like forms seemed not to develop further, but a few EEF-like forms showed significant increase in size and compartmentalization of HSP70 staining. This change might correspond to the cytoplasmic compartmentalization observed in intracellular EEF at this time point (Meis et al. Parasitology 1981; 82:195-204). These HSP70 staining patterns closely resembled the staining patterns observed for intracellular EEF developing inside HepG2 cells.

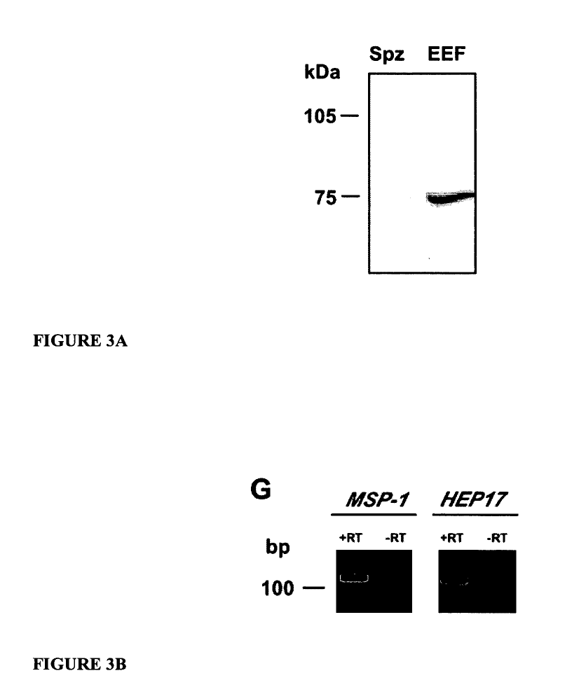

Upregulation of HSP70 expression was confirmed by immunoblot analysis (FIG. 3A). HSP70 expression was barely detectable in protein extracts of 800,000 salivary gland sporozoites. However, expression increased dramatically when the same number of parasites were cultured in the host cell free system for 24 hours (13% transformation rate). HSP70 expression might be induced by temperature elevation, the transformation event itself, or both. However, because parasites kept at 37° C. for 24 hours either transformed or died it was not possible to investigate this further.

CSP, the major sporozoite surface protein (Aikawa et al. J. Immunol. 1981; 126:2494-2495) is involved in sporozoite host cell recognition. It is also expressed on the plasma membrane of early EEFs (Danforth et al. J Parasitol 1978; 64:1123-1125 and Hamilton et al. Cell Biol Int Rep 1988; 12:123-129) and might fulfill additional functions during liver stage development. Robust CSP surface expression was detected on spherical EEFs, generated by culture of sporozoites in the absence of host cells for 24 hours, using a monoclonal antibody specific for CSP. In contrast, TRAP, a sporozoite-specific micronemal protein involved in host cell invasion (Sultan et al. Cell 1997; 90:511-522), was not detectable on spherical EEFs forms at this time. The loss of TRAP expression coincides with the disappearance of micronemes after sporozoite transformation in vivo (Meis et al. Cell Tissue Res 1985; 241:353-360).

EEFs formed by culture of sporozoites in the absence of host cells for 24 hours were examined for expression of transcripts encoding the merozoite surface protein-1 (MSP-1) (Suhrbier et al. Am J Trop Med Hyg 1989; 40:351-355) and the hepatic and erythrocytic stage protein 17 (HEP17) (Charoenvit et al. Exp Parasitol 1995; 80:419-429) via RT-PCR. The P. berghei spherical EEFs, generated by culture of sporozoites in the absence of host cells for 24 hours, expressed transcripts for both MSP-1 and HEP17 (FIG. 3B).

Apicomplexan zoites, including the Plasmodium sporozoite are delimited by a tri-laminar pellicle consisting of a plasma membrane and two closely aligned inner membranes that form the inner membrane complex (IMC). After hepatocyte invasion the sporozoite plasma membrane becomes the EEF plasma membrane, however, the IMC is disassembled and is not detectable at approximately 30 hours post invasion (Meis et al. Cell Tissue Res 1985; 241:353-360). Dismantling the IMC might be essential for entry of the parasite into the trophic phase because its rigidity could interfere with growth. MTIP localizes to the inner membrane complex of sporozoites (Bergman et al. J Cell Sci. 2003; 116:39-49) and can serve as a marker to follow the loss of the IMC during liver stage development.

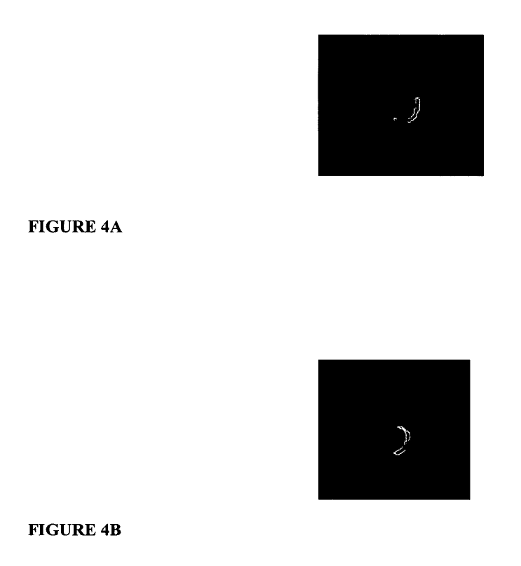

Dual fluorescence assays with antibodies against CSP and MTIP showed that the IMC was lost in EEFs generated by culture of sporozoites in the absence of host cells for 24 hours. IMC loss was sometimes simultaneous with a first round of nuclear division but these events did not occur in a synchronized fashion. The IMC loss in host cell-free EEFs (FIG. 4A) closely resembled the IMC loss observed for intracellular EEFs cultures with HepG2 host cells (FIG. 4B).

Inclusion of lactacystin (10 μM), a highly specific inhibitor of proteasome activity, in the culture medium inhibited host cell-free transformation by ˜70% indicating that host-cell-free EEF transformation is a proteasome-dependent process. This was consistent with its previously observed inhibitory effect on sporozoite transformation into intracellular EEF (Gantt et al. Antimicrob Agents Chemother 1998; 42:2731-2738).

Taken together, the data show that, in the absence of intact host cells or host cell derived components, sporozoites transform into EEF that undergo nuclear division and express proteins similar to early EEF that develop within host cells.

The host cell-free culture system provides a unique opportunity to study what environmental factors that directly govern transformation. The stimulus for transformation might be a shift in temperature experienced by the parasite during transmission. Sporozoites were tested at either 22° C. (temperature of mosquito vector) or 37° C. (temperature of mammalian host) for 24 hours. In contrast to sporozoites cultured at 37° C., sporozoites cultured at 22° C. initiated transformation (revealed by the occurrence of transformation bulbs) but rarely developed into completely spherical EEF (see Table 1). Therefore a shift from low to high temperature is not necessary for initiation of the transformation process, but high temperature is required for complete transformation of sporozoites into EEF. Interestingly, parasites kept at 22° C. for 24 hours could still develop into spherical EEF when the temperature was subsequently shifted to 37° C. for 24 hours (Table 1), and this occurred with higher efficiency of transformation (˜19%) than in cultures directly incubated at 37° C. Thus parasites kept at low temperature remained viable, experiencing a reversible arrest of transformation. In addition, the presence of serum was also found to be critical for transformation (Table 1). Without serum few parasites transformed into spherical EEF and most parasites were not detectable in 37° C. cultures after 24 hours.

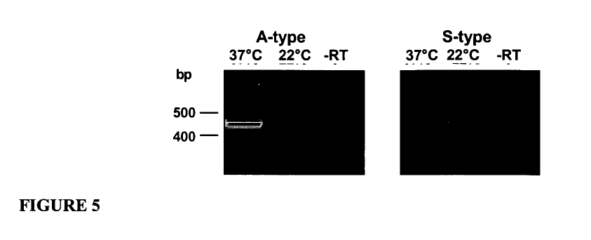

Switching occurs between the expression of different types of ribosomal RNA (rRNA) at transition points in the Plasmodium life cycle (Gunderson et al. Science 1987; 238:933-937). One such transition is the transformation of sporozoites into EEFs when rRNA expression switches from S-type in sporozoites (S for sporozoite) to A-type in EEFs and succeeding blood stages (A for Asexual) (Zhu et al. J Biol. Chem. 1990; 265:12740-12744). rRNA expression was determined in the host cell-free transformation system at 22° C. and 37° C. after 24 hours of culture by reverse transcriptase-PCR using oligonucleotide primers specific for either A-type or S-type rRNA. Expression of A-type rRNA was detected in host cell-free cultures at both temperatures (FIG. 5). Thus expression of A-type rRNA was not dependent on temperature elevation, however it seemed to increase with the elevated temperature. S-type rRNA was detectable at 22° C. but was not detectable at 37° C. confirming the rRNA switch (FIG. 5). This indicated that either the elevated temperature repressed expression of S-type rRNA, or that expression of S-type rRNA is under control of a cold-stimulated promoter. Thermoregulation of rRNA gene expression was recently described to also occur in the parasites' blood stages (Fang and McCutchan. Nature 2002; 418:742).

A central tenet of Plasmodium transmission is that the invasive sporozoite must penetrate a host hepatocyte and take up intracellular residence to initiate development into EEF. However, these results show that, in principle, sporozoites do not require an intact host cell or any specific host cell-derived factors to transform into EEF. In vivo, after sporozoites enter the blood stream they rapidly sequester in the liver and invade hepatocytes within minutes (Shin et al. J Protozool. 1982; 29:448-454). This rapid homing ensures that transformation occurs only after invasion of a suitable host cell. Although sporozoites can transform into EEF without host cells, intracellular residence is likely to be essential for the parasites trophic phase when host cell-derived factors are needed for further growth. Plasmodium merozoites can transform into trophozoites and develop outside a red blood cell (Trager et al. Proc Natl Acad Sci USA 1990; 87:5618-5622) only in the presence of red cell extract, indicating that merozoite transformation and subsequent parasite development was dependent on some yet undefined host cell factors.

### Example 2

### A Plasmodium Axenic Liver Stage Vaccine Provides Protective Immunity

### Materials and Methods

Malaria parasites: Plasmodium yoelii sporozoites were collected in batches of 5 to 6 million sporozoites as described in Example 1. Plasmodium yoelii is the infectious agent in rodent malaria.

Transformation medium and quantification of transformation: Sporozoites were suspended in DMEM medium (BioWhittaker) containing 2 mM of L-glutamine, 4.5 g/l glucose and supplemented with 10% Fetal Bovine Serum (FBS; HyClone) and 500 U/ml penicillin/streptomycin and 1.25 μl/ml fungizon (Sigma). Various amounts of sporozoites were inoculated per well of a 6 well chamber slide (Labtek) and maintained at 37° C. in 5% CO₂ for 24 hours. The transformed Plasmodium yoelii EEFs (Plasmodium yoelii axenic liver stages) were then collected. These collected Plasmodium yoelii axenic liver stages also contained the un-separated cellular debris from the untransformed parasites that died during the culture period.

Transformation percentages for various culture batches were determined by immunofluorescence assay as described in Example 1 using a monoclonal antibody against CSP (Circumsporozoite Protein) (antibody generated as described in Yoshida et al. Science 1980; 207:71-73) and a rabbit polyclonal antibody against MTIP (Myosin A tail domain interacting protein) (antibody generated as described in Bergman et al. J. Cell. Sci. 2003; 116:39-49).

Immunization protocol: Groups of Balb/c mice were injected subcutaneously with different doses of Plasmodium yoelii axenic liver stages (see Table 2). Mice were injected on the following schedule: primary injection on day 1, first boost injection on day 14, and second boost injection on day 21. Seven days after the second boost injection, the vaccinated mice were then challenged by subcutaneous injection of 10,000 Plasmodium yoelii sporozoites. Then 44 hours after challenge, the livers of the mice were collected and subjected to quantitative real-time RT-PCR to determine liver-stage burden (i.e., the number of liver stage parasites in the liver).

Quantitative real-time reverse transcriptase (RT)-PCR: The RNA of harvested livers was isolated by phenol chloroform extraction using Trizol reagent (Invitrogen) according to the manufacturer's instructions. The isolated RNA was treated with DNAse I (Invitrogen) to remove contaminating genomic DNA. First strand cDNA synthesis was performed according to manufacturer's instructions using the Taqman RT reagent kit (Applied Biosystems) with random hexamers. Control reactions were performed in the absence of reverse transcriptase.

The reverse transcribed RNA then served as template for a quantitative real-time PCR. This PCR was used to quantitate the level of Plasmodium yoelii liver stage 18S rRNA transcripts relative to the level of endogenous mouse GAPDH transcripts as an internal control.

A reaction containing a defined copy number of plasmid PY18S, which contains the nucleotide sequence of Plasmodium yoelii liver stage 18S rRNA (Genbank accession number AF180727) inserted in the pCRe4-TOPO® vector (Invitrogen) served as the 18S rRNA copy-number control. These reactions contained amount of plasmid DNA corresponding to an 18S rRNA copy number of from 10⁶ to 10³.

A reaction containing a defined copy number of the mouse GAPDH standard which contains the nucleotide sequence of mouse GAPDH (Genbank Accession number M32599) inserted in the pCR®4-TOPO® vector (Invitrogen) served as the GAPDH standard copy number control. These reactions contained amount of plasmid DNA corresponding to a mouse GAPDH copy number of from 10⁶ to 10³.

PCR amplification of 18S rRNA was performed using primers PY18Ssense (5′-GGG GAT TGG TTT TGA CGT TTT TGC G-3′; SEQ ID NO: 9) and PY18Santisense (5′-AAG CAT TAA ATA AAG CGA ATA CAT CCT TA-3′; SEQ ID NO: 10) which amplify a specific 133 bp fragment.

PCR amplification of GAPDH was performed using primers GAPDHsense (5′-CCT CAA CTA CAT GGT TTA CAT-3′; SEQ ID NO: 11) and GAPDHantisense (5′-GCT CCT GGA AGA TGG TGA TG-3′; SEQ ID NO: 12), which amplify a specific 122 bp fragment.

Quantitative real-time PCR was performed in 96-well plates where each well contained a 50 μL reaction mixture of Supermix BioRad 2× (BioRad) reagent, 125 picomoles of forward primer, 125 picomoles of reverse primer, and 4 μL of template (either reverse transcribed RNA or copy number control). Each sample of reverse transcribed RNA was amplified in triplicate with each primer set (for a total of 6 PCR reactions). Amplification was performed using the iCycler PCR machine (BioRad) using the following program: 95° C. for 3 minutes then 40 cycles of 95° C. for 30 seconds and 60° C. for 30 seconds.

Quantification was obtained by detection and measurement in real time of the SYBR green I dye emitted fluorescence proportionally to the synthesis of the PCR product.

The liver-stage infection burden was determined by normalization of level of P. yoelii 18S rRNA product for a given reaction to the level of murine GAPDH product in the corresponding reaction.

The three normalized values for liver-stage infection burden product for a liver sample for a given mouse were then averaged (expressed as mean±standard deviation). In the case of the 5 naïve mice of the control group, these 5 individual normalized values were then averaged to give a combined control liver-stage infection burden (expressed as mean±standard deviation).

The average normalized value for 18S product in the liver of each experimental mouse (vaccinated with Plasmodium yoelii axenic liver stages) was then compared to the combined control 18S product level value to calculate the relative inhibition of liver-stage burden for each experimental mouse (expressed as percent inhibition). The relative inhibition values for mice of the same vaccination group were then averaged to calculate an average relative inhibition percentage (expressed as mean±standard deviation). These average relative inhibition percentages were then plotted in a bar graph versus vaccination dose of Plasmodium yoelii axenic liver stages.

### Results and Discussion

The ability of immunization with vaccines of Plasmodium axenic liver stages to confer protective immunity against malarial infection was assessed in a mouse model. The vaccines comprised various amounts of Plasmodium yoelii axenic liver stages generated by culturing sporozoites, without host cells, in medium containing 10% FBS at 37° C. for 24 hours.

Groups of Balb/c mice were injected subcutaneously with different doses of Plasmodium yoelii axenic liver stages (see Table 2). Note that for each injected dose, the indicated number of total parasites was used to innoculate the host-cell free transformation culture. After 24 hours of culture, the indicated number of live Plasmodium yoelii axenic liver stages remained in the culture. These live Plasmodium yoelii axenic liver stages were then injected into the mice. Note that these injections also contained some cellular debris from the untransformed parasites that died during the 24 hour transformation culture period. The efficiency of transformation for the host-cell free cultures used to generate the Plasmodium yoelii axenic liver stages for the primary and first and second boost injections is given in Table 3.

TABLE 2
Immunization groups and doses of Plasmodium yoelii axenic liver stages

| | Injected doses | |
|---|---|---|
| Number of mice | Total parasites* | Dose of axenic liver stages^{†} |
| 5 | 1,000,00 | 100,000 |
| 5 | 200,000 | 20,000 |
| 6 | 40,000 | 4,000 |
| 6 | 8,000 | 800 |
| 5 | Negative control^{‡} | Negative control^{‡} |

*Figure indicates the total number of sporozoites inoculated into the host-cell free transformation culture used to generate live transformed Plasmodium yoelii axenic liver stages for injection.
^{†}Figure indicates the approximate number of live transformed Plasmodium yoelii axenic liver stages in each injection.
^{‡}Negative control injections contained the culture medium from cultures of uninfected mosquito salivary glands.
These mice served as the naïve controls for subsequent challenge.

TABLE 3
Efficiency of transformation for the host-cell free cultures
used to generate Plasmodium yoelii axenic liver stages

| Injection batch | Transformation efficiency^{‡} |
|---|---|
| Primary injection | 10.0% |
| First boost injection | 13.7% |
| Second boost injection | 9.0% |

^{‡}Percentage calculated as (# of EEFs/# of sporozoites inoculated into culture) × 100.

Mice were injected on the following schedule: primary injection on day 1, first boost injection on day 14, and second boost injection on day 21.

Seven days after the second boost injection, the vaccinated mice were then challenged by subcutaneous injection of 10,000 Plasmodium yoelii sporozoites. Then 44 hours after challenge, the livers of the mice were collected. The RNA from each liver was isolated and subjected to quantitative real-time RT-PCR to determine liver-stage burden (i.e., the number of Plasmodium yoelii liver stages in the liver).

Reverse transcribed RNA isolated from the livers of challenged, vaccinated mice was subjected to quantitative real-time PCR to measure the level of Plasmodium yoelii liver stage 18S rRNA transcripts present in the isolated RNA. In this analysis, the level of Plasmodium yoelii liver stage 18S rRNA transcripts in a given RNA sample is directly proportional to the liver-stage burden (i.e., the number of Plasmodium yoelii liver stages in the liver) of the liver from which the RNA was isolated. Thus, a decrease in the level of 18S transcripts in a vaccinated mouse, relative to a control naïve mouse, indicates that the liver-stage burden is reduced. Any reduction in liver-stage burden in a vaccinated mouse reflects protective immunity conferred by immunization with the Plasmodium yoelii axenic liver stage vaccine.

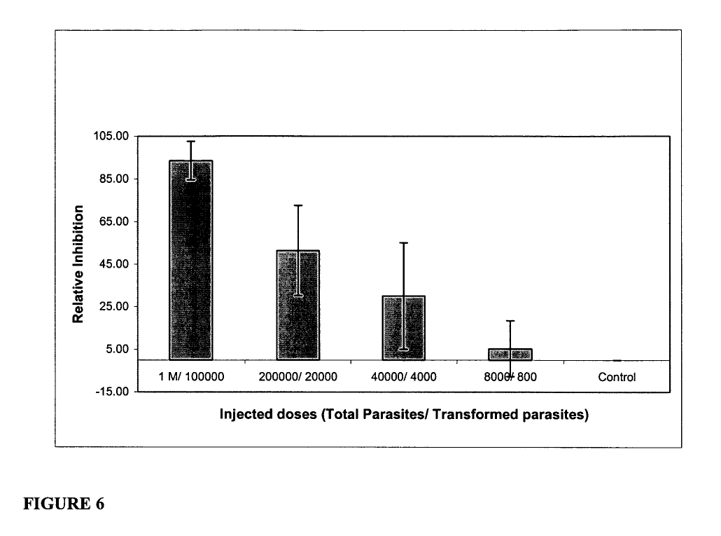

This experiment showed that vaccination with live Plasmodium yoelii axenic liver stages conferred protective immunity to subsequent malarial infection (FIG. 6). Immunization (three times) with a dose of 100,000 Plasmodium yoelii axenic liver stages provided near complete protection, as shown by 94% inhibition of liver-stage burden. Immunization (three times) with doses of 20,000 or 4,000 Plasmodium yoelii axenic liver stages was also substantially protective, as shown by 51% and 30% inhibition of liver-stage burden, respectively.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method for the generation of a Plasmodium axenic liver stage, which method comprises culturing a Plasmodium sporozoite in the absence of a hepatocyte or a hepatocyte-derived factor, wherein the Plasmodium sporozoite is cultured at a temperature ranging between about 35° C. and about 39° C. in culture medium comprising serum.

2. The method of claim 1, wherein the temperature is about 37° C.

3. The method of claim 1, wherein the culture medium comprises between about 5% and about 15% by volume of serum.

4. The method of claim 3, wherein the culture medium comprises about 10% by volume of serum.

5. The method of claim 1, wherein the serum is fetal bovine serum.

6. The method of claim 1, wherein the culture medium is DMEM.

7. The method of claim 1, wherein the Plasmodium sporozoite is cultured at a temperature of about 37° C. for at least about 18 hours.

8. The method of claim 7, wherein the Plasmodium sporozoite is cultured at a temperature of about 37° C. for about 24 hours.

9. The method of claim 1, wherein the Plasmodium sporozoite is cultured at a temperature between about 18° C. and about 24° C. for up to about 24 hours prior to culture at between about 35° C. to between about 39° C.

10. The method of claim 9, wherein the sporozoites are cultured at about 22 C.

10. The method of claim 9, wherein the sporozoites are cultured at about 22° C.

11. The method of claim 9, wherein the sporozoites are cultured for about 24 hours.

12. The method of claim 9, wherein the reduced temperature is about 22° C. and the sporozoites are cultured for about 24 hours.

13. The method of claim 1, wherein the Plasmodium sporozoite is a Plasmodium falciparum sporozoite.

14. An isolated Plasmodium axenic liver stage sporozoite substantially free of hepatocytes proteins or hepatocytes surface markers produced by the method of claim 1.

15. The Plasmodium axenic liver stage of claim 14, wherein the Plasmodium axenic liver stage is a Plasmodium falciparum axenic liver stage.

16. An isolated Plasmodium axenic liver stage sporozoite produced by the method of claim 12.

17. The Plasmodium axenic liver stage of claim 16, wherein the Plasmodium axenic liver stage is a Plasmodium falciparum axenic liver stage.

18. A method for the prevention or treatment of malaria, which comprises administering to a subject in need of such prevention or treatment a vaccine comprising a therapeutically effective amount of Plasmodium axenic liver stage substantially free of hepatocyte proteins.

19. The method of claim 18, wherein the Plasmodium axenic liver stage is a Plasmodium falciparum axenic liver stage.

20. The method of claim 18, wherein the subject is a human.

21. A vaccine for the prevention or treatment of malaria comprising a Plasmodium axenic liver stage substantially free of hepatocytes proteins and a pharmaceutically acceptable carrier.

22. The vaccine of claim 21, further comprising an adjuvant.

23. The vaccine of claim 21, wherein the Plasmodium axenic liver stage is a Plasmodium falciparum axenic liver stage.


SEQUENCE LISTING (12 sequences)

SEQ ID NO: 1 - DNA, 22 bp, artificial
  primer
cgagaatcttggctccgcctcg

SEQ ID NO: 2 - DNA, 24 bp, artificial
  primer
ctaagaaatccccgaagggaaatc

SEQ ID NO: 3 - DNA, 25 bp, artificial
  primer
catgacttctgtcactgcttttatc

SEQ ID NO: 4 - DNA, 23 bp, artificial
  primer
ctactcctttaaagaagatagtt

SEQ ID NO: 5 - DNA, 20 bp, artificial
  primer
ggagaaaatgcagtggtaag

SEQ ID NO: 6 - DNA, 29 bp, artificial
  primer
ttttaaatgcctcaagaatatttttttct

SEQ ID NO: 7 - DNA, 20 bp, artificial
  primer
aaaagggaagacatccattc

SEQ ID NO: 8 - DNA, 21 bp, artificial
  primer
tgtattccttcggatgaaaaa

SEQ ID NO: 9 - DNA, 25 bp, artificial
  primer
ggggattggttttgacgtttttgcg

SEQ ID NO: 10 - DNA, 29 bp, artificial
  primer
aagcattaaataaagcgaatacatcctta

SEQ ID NO: 11 - DNA, 21 bp, artificial
  primer
cctcaactacatggtttacat

SEQ ID NO: 12 - DNA, 20 bp, artificial
  primer
gctcctggaagatggtgatg